(19) United States
(12) Patent Application Publication
(10) Pub. No.: US 2009/0098167 A1
(43) Pub. Date: Apr. 16, 2009

(54) PHL P 1 Allergen Derivative

(75) Inventors: Tanja Ball, Vienna (AT); Birgit Linhart, Weissenkirchen (AT); Peter Valent, Vienna (AT); Angelika Stocklinger, Salzburg (AT); Christian Lupinek, Vienna (AT); Josef Thalhamer, Lamprechtshausen (AT); Rudolf Valenta, Theresienfeld (AT)

Correspondence Address: CHALKER FLORES, LLP, 2711 LBJ FRWY, Suite 1036, DALLAS, TX, 75234, US

(73) Assignees: BIOMAY AG, Vienna (AT); ALLERGOPHARMA JOACHIM GANZER KG, Reinbek (DE)

(21) Appl. No.: 12/299,292

(22) PCT Filed: May 3, 2007
(86) PCT No.: PCT/AT2007/000208

§ 371 Date: Oct. 31, 2008

(30) Foreign Application Priority Data
May 3, 2006 (AT) A 755/2006

(51) Int. Cl. A61K 39/36; C07K 14/415; A61P 37/08; C12P 21/02

(52) U.S. Cl. 424/275.1; 530/379; 435/69.1

(57) ABSTRACT

Method for producing derivatives of wild-type protein allergen Phl p 1 with reduced allergenic activity compared to the wild-type allergen, comprising the following steps: providing wild-type protein allergen Phl p 1, fragmenting said wild-type protein allergen into at least three fragments, wherein at least one fragment of said at least three fragments comprises at least one T-cell epitope and said at least three fragments have a reduced allergenic activity or lack allergenic activity and rejoining said at least three fragments in an order differing from the order of the fragments in the wild-type allergen.

The present invention relates to derivatives of wild-type protein allergen Phl p 1 and methods for producing them.

Allergy is the inherited or acquired specific alternation of the reaction capability against foreign (i.e. non-self) substances which are normally harmless (“allergens”). Allergy is connected with inflammatory reactions in the affected organ systems (skin, conjunctiva, nose, pharynx, bronchial mucosa, gastrointestinal tract), immediate disease symptoms, such as allergic rhinitis, conjunctivitis, dermatitis, anaphylactic shock and asthma, and chronic disease manifestations, such as late stage reactions in asthma and atopic dermatitis.

Type I allergy represents a genetically determined hyper-sensitivity disease which affects about 20% of the industrialised world population. The pathophysiological hallmark of Type I allergy is the production of immunoglobulin E (IgE) antibodies against otherwise harmless antigens (allergens).

Currently, the only causative form of allergy treatment is an allergen-specific immunotherapy wherein increasing allergen doses are administered to the patient in order to induce allergen-specific unresponsiveness. While several studies have shown clinical effectiveness of allergen-specific immunotherapy, the underlying mechanisms are not fully understood.

The major disadvantage of allergen-specific immunotherapy is the dependency on the use of natural allergen extracts which are difficult, if not impossible to standardise, at least to an industrial production level. Such natural allergen extracts consist of different allergenic and non allergenic compounds and due to this fact it is possible that certain allergens are not present in the administered extract or—even worse—that patients can develop new IgE-specificities to components in the course of the treatment. Another disadvantage of extract-based therapy results from the fact that the administration of biologically active allergen preparations can induce anaphylactic side effects.

The application of molecular biology techniques in the field of allergen characterisation has allowed to isolate the cDNAs coding for all relevant environmental allergens and allowed the production of recombinant allergens. Using such recombinant allergens has made it possible to determine the individual patient's reactivity profile either by in vitro diagnostic methods (i.e. detection of allergen-specific IgE antibodies in serum) or by in vivo testing. Based on this technology, the possibility to develop novel component-based vaccination strategies against allergy, especially against Type I allergy, which are tailored to the patient's sensitisation profile appeared to be possible. However, due to the similarity of the recombinant allergens to their natural counterparts, also recombinant allergens exhibit significant allergenic activity. Since the recombinant allergens closely mimick the allergenic activity of the wild-type allergens, all the drawbacks connected with this allergenic activity in immunotherapy applying natural allergens are also present for recombinant allergens. In order to improve immunotherapy the allergenic activity of the recombinant allergens has to be reduced so that the dose of the administered allergens can be increased with only a low risk of anaphylactic side effects.

It has been suggested to influence exclusively the activity of allergen-specific T cells by administration of peptides containing T cell epitopes only. T cell epitopes represent small peptides which result from the proteolytic digestion of intact allergens by antigen representing cells. Such T cell epitopes can be produced as synthetic peptides. Tests conducted so far with T cell epitopes, however, only showed poor results and low efficacy. Several explanations for the low efficacy of T cell peptide-based immunotherapy have been considered: first, it may be difficult to administer the optimal dose to achieve T cell tolerance instead of activation. Second, small T cell epitope peptides will have a short half-life in the body. Third, there is considerable evidence that IgE production in atopic individuals represents a memory immune response which does not require de novo class switching and thus cannot be controlled by T cell-derived cytokines. Therapy forms which are based exclusively on the administration of T cell epitopes may therefore modulate the activity of allergen-specific T cells but may have little influence on the production of allergen-specific IgE antibodies by already switched memory B cells.

It has further been suggested to produce hypoallergenic allergen derivatives or fragments by recombinant DNA technology or peptide synthesis. Such derivatives or fragments bear T cell epitopes and can induce IgG antibodies that compete with IgE recognition of the native allergen. It was demonstrated more than 20 years ago that proteolytic digestion of allergens yielded small allergen fragments which in part retained their IgE binding capacity but failed to elicit immediate type reactions. While proteolysis of allergens is difficult to control and standardise, molecular biology has opened up new avenues for the production of IgE binding haptens. Such IgE binding haptens have been suggested to be useful for active immunisation with reduced risks of anaphylactic effects and for passive therapy to saturate effector cell-bound IgE prior to allergen contact and thus block allergen-induced mediator release.

Another suggestion was to produce hypoallergenic allergen versions by genetic engineering based on the observation that allergens can naturally occur as isoforms with differ in only a few amino acid residues and/or in conformations with low IgE binding capacity. For example, oligomerisation of the major birch pollen allergen, Bet v 1, by genetic engineering yielded a recombinant trimer with greatly reduced allergenic activity. Alternatively, introduction of point mutations has been suggested to either lead to conformational changes in the allergen structure and thus disrupt discontinuous IgE epitopes or directly affect the IgE binding capacity (Valenta et al., Biol. Chem.380 (1999), 815-824).

It has also been shown that fragmentation of the allergen into few parts (e.g. into two parts) leads to an almost complete loss of IgE binding capacity and allergenic activity of the allergen due to a loss of their native-like folds (Vrtala et al. J. Clin. Invest. 99 (1997), 1673-1681) for Bet v 1, Twardosz et al. (BBRC 239 (1997), 197-204) for Bet v 4, Hayek et al. (J. Immunol. 161 (1998), 7031-7039) for Aln g 4, Zeiler et al. (J. Allergy Clin. Immunol. 100 (1997), 721-727) for bovine dander allergen, Elfman (Int. Arch. Allergy Immunol. 117 (1998), 167-173) for Lep d2), Westritschnig (J. Immunol. 172 (2004), 5684-5692) for Phlp 7)). Fragmentation of proteins containing primarily discontinuous/conformational IgE epitopes leads to a substantial reduction of the allergen's IgE binding capacity. Based on this knowledge, it was investigated in the prior art whether such hypoallergenic allergen fragments can induce protective immune responses in vivo (Westritschnig et al. (Curr. Opinion in Allergy and Clin. Immunol. 3 (2003), 495-500)).

In Ball T et al. (Eur J Immunol 1999, 29: 2026-2036) the administration of grass pollen extracts adsorbed through aluminim hydroxide in the course of an immunotherapy is described.

In the US 2002/0052490 A1 recombinant DNA molecules coding for a polypeptide which comprises at least one Phl P 1-epitope are disclosed.

In Flicker S et al. (J Allergy Clin Immunol 2006, 117: 1336-1343) it was discovered that a C-terminal art of the Phl p 1 allergen comprises most of the allergenic potential of the whole Phi p 1 molecule.

In Linhart B et al. (FASEB J 2002, 16: 1301-1303) the production of a hybride molecule comprising several allergens of Timothy grass is described.

DE 103 51 471 A1 concerns hybride polypeptides consisting of several immuno-dominant T-cell epitopes of allergens, which do not cross react.

It is an object of the present invention to provide means and methods for improved allergy immunotherapy based on the above mentioned knowledge. Such methods and means should be effective, connected with a low risk for anaphylactic shock, easily applicable and adapted to the needs of an individual patient and easily transformable into industrial scales.

Therefore the present invention relates to a method for producing derivatives of wild-type protein allergen Phl p 1 (a major pollen allergen of Timothy grass) with reduced allergenic activity compared to the wild-type allergen, comprising the following steps:
providing wild-type protein allergen Phl p 1,
fragmenting said wild-type protein allergen into at least three fragments, wherein at least one fragment of said at least three fragments comprises at least one T-cell epitope and said at least three fragments have a reduced allergenic activity or lack allergenic activity and
rejoining said at least three fragments in an order differing from the order of the fragments in the wild-type allergen.

Allergic reactions are triggered when allergens cross-link preformed IgE bound to the high-affinity receptor FcεRI on mast cells. Mast cells line the body surfaces and serve to alert the immune system to local infection. Once activated, they induce inflammatory reactions by secreting chemical mediators stored in preformed granules and by synthesizing leukotrienes and cytokines after activation occurs. Therefore, the administration of wild-type allergens in order to prevent, treat or sensitise individuals suffering or at risk for suffering an allergy is not advantageous due to side effects caused. A way to avoid allergic reactions in immunotherapy is to modify the wild-type allergen to such an extent that said modified “allergens” bind to IgE in a reduced amount or not any more. Consequently such molecules are not able provoke a strong allergic reaction. However, it should be noted that fragments of certain allergens may be not enough immunogenic to induce a protective antibody response (Westritschnig et al., (2004)).

The method according to the present invention allows the production of allergen derivatives with a reduced or missing capacity to bind to IgE but conserving at the same time those features of the allergen which are required to induce a T cell mediated immune response. This can be achieved with the method according to the present invention, because those structural elements, which are responsible for the induction of a T cell mediated immune response, e.g. T cell epitopes of the wild-type allergen, will remain substantially conserved in the allergen derivative according to the present invention. However, a shuffling (fragmenting and rejoining) of the fragments of the allergen will lead to a significant reduction of the IgE binding capacity or even to a complete loss of said binding capacity. Of course, if only a few amino acid residues are lost (deleted) or added (inserted) in the course of generation of the allergen derivatives or if the parts are combined by a linker instead of a direct combination, the advantages according to the present invention are still present. This reduction or abolishment of allergenic activity is achieved by the known and general principle of dividing the allergen into defined fragments.

The allergen derivative according to the present invention is preferably produced recombinantly. Of course it is also possible to synthesize the single allergen fragments chemically and then to link them together.

According to a preferred embodiment of the present invention the reduction in allergenic activity is measured by a reduction of IgE binding capacity of at least 10%, preferably at least 20%, more preferably at least 30%, especially at least 50%, compared to the wild-type allergen.

The reduction in allergenic activity is measured preferably by lack of binding of IgE antibodies of allergen sensitised patient's sera to a dot blot of said derivative or by a basophil release assay.

Conventional in vitro assays for assessing allergenic activity include RAST (Sampson and Albergo, J. Allergy Clin. Immunol. 74:26, 1984), ELISAs (Burks et al., N. Engl. J. Med. 314:560, 1986), immunoblotting (Burks et al., J. Allergy Clin. Immunol. 81:1135, 1988), basophil histamine release assays (Nielsen, Dan. Med. Bull. 42:455, 1995 and du Buske, Allergy Proc. 14:243, 1993) and others (Hoffmann et al., Allergy 54:446, 1999).

According to a preferred embodiment of the present invention the at least three fragments comprise amino acid residues 25 to 39, amino acid residues 34 to 45, amino acid residues 73 to 84, amino acid residues 91 to 102, amino acid residues 100 to 111, amino acid residues 109 to 133, amino acid residues 121 to 135, amino acid residues 127 to 138, amino acid residues 157 to 168, amino acid residues 169 to 183 and/or amino acid residues 226 to 240 of Phl p 1.

The at least three fragments to be used in the method according to the present invention may comprise at least one of the above identified T cell epitopes (see e.g. Schenk S, et al. J Allergy Clin Immunol. (1995) 96:986-996).

According to another preferred embodiment of the present invention the at least three fragments are selected from the group consisting of amino acid residues 1 to 64 (A), amino acid residues 65 to 125 (B), amino acid residues 126 to 205 (C) and amino acid residues 206 to 240 (D) of Phl p 1.

The fragments of the present invention are selected in order to destroy IgE binding/B cell epitopes and conserving T cell epitopes which may induce the production of an appropriate immune response to said epitopes. The destruction/reduction of B cell epitopes naturally present in the wild-type allergen Phl p 1 may allow that said derivatives mainly provoke a T cell mediated response when administered to an individual rather than induce an allergic reaction (complete lack or reduced binding to IgE bound to mediator releasing cells).

The order of said fragments in the allergen derivative is preferably B-D-A-C.

Of course it is also possible to order said fragments in an alternative manner (D-B-A-C, B-A-D-C etc.), provided that the allergen derivative obtained exhibits a reduced allergenic activity as compared to the wild-type allergen.

The derivatives obtained according to the present invention may be easily combined with a pharmaceutically acceptable excipient and finished to a pharmaceutical preparation.

Preferably, the derivatives are combined with a suitable vaccine adjuvant and finished to a pharmaceutically acceptable vaccine preparation.

According to a preferred embodiment, the derivatives according to the present invention are combined with further allergens to a combination vaccine. Such allergens are preferably wild-type allergens, especially a mixture of wild-type allergens, recombinant wild-type allergens, derivatives of wild-type protein allergens or mixtures thereof. Such mixtures may be made specifical for the needs (allergen profile) of a certain patient.

In a preferred embodiment, such a pharmaceutical preparation further contains an allergen extract.

According to another preferred embodiment of the present invention said further allergen is selected from the group consisting of the major birch pollen allergens, in particular Bet v 1 and Bet v 4, the major timothy grass pollen allergens, in particular Phl p 2, Phl p 5, Phl p 6 and Phl p 7, the major house dust mite allergens, in particular Der p 1 and Der p 2, the major cat allergen Fel d 1, the major bee allergens, the major wasp allergens, profilins, especially Phl p 12, and storage mite allergens, especially Lep d 2.

The pharmaceutical and vaccine preparations according to the present invention may comprise next to a derivative of Phl p 1 other allergens or derivatives and fragments thereof, allergen extracts etc.

Another aspect of the present invention relates to an allergen derivative obtainable by a method according to the present invention.

Yet another aspect of the present invention relates to an allergen derivative of wild-type protein allergen Phl p 1, characterized in that said derivative contains at least three fragments of said wild-type protein allergen fused to each other in an order differing from the order of the fragments in the wild-type allergen, wherein said at least three wild-type allergen fragments exhibit reduced allergenic activity or lack allergenic activity and wherein at least one of said at least three fragments comprises one or more T cell epitopes.

According to a preferred embodiment of the present invention said at least three allergen fragments comprise at least 6 amino acid residues, preferably at least 10 amino acid residues, especially at least 15 amino acid residues.

The at least three fragments comprise preferably amino acid residues 25 to 39, amino acid residues 34 to 45, amino acid residues 73 to 84, amino acid residues 91 to 102, amino acid residues 100 to 111, amino acid residues 109 to 133, amino acid residues 121 to 135, amino acid residues 127 to 138, amino acid residues 157 to 168, amino acid residues 169 to 183 and amino acid residues 226 to 240 of Phl p 1.

According to another preferred embodiment of the present invention the at least three fragments are selected from the group consisting of amino acid residues 1 to 64 (A), amino acid residues 65 to 125 (B), amino acid residues 126 to 205 (C) and amino acid residues 206 to 240 (D) of Phl p 1.

The order of said fragments in the allergen derivative is preferably B-D-A-C.

The allergen derivatives of the present invention can also be used for detecting and diagnosing sensitivity to Phl p 1. For example, this can be done in vitro by combining blood or blood products obtained from a subject to be assessed for sensitivity with a peptide having an activity of Phl p 1, under conditions appropriate for binding of components in the blood (e.g. antibodies, T cells, B cells) with the derivatives and determining the extent to which such binding occurs. Other diagnostic methods for allergic diseases with which the derivatives of the present invention can be used include radio-allergosorbent test (RAST), paper radioimmunosorbent test (PRIST), enzyme linked imununosorbent assay (ELISA), radioimmunoassays (RIA), immunoradiometric assays (IRMA), luminescence immunoassays (LIA), histamine release assays and IgE immunoblots.

Another aspect of the present invention relates to an allergen composition comprising an allergen derivative according to the present invention (see above) and further allergens, preferably wild-type allergens, especially a mixture of wild-type allergens, recombinant wild-type allergens, derivatives of wild-type protein allergens or mixtures thereof.

Said composition further contains preferably an allergen extract.

According to a preferred embodiment of the present invention the allergen composition contains a pharmaceutically acceptable excipient.

According to another preferred embodiment of the present invention said composition further comprises one or more allergens selected from the group consisting of the major birch pollen allergens, in particular Bet v 1 and Bet v 4, the major timothy grass pollen allergens, in particular Phl p 2, Phl p 5, Phl p 6 and Phl p 7, the major house dust mite allergens, in particular Der p 1 and Der p 2, the major cat allergen Fel d 1, the major bee allergens, the major wasp allergens, profilins, especially Phi p 12, and storage mite allergens, especially Lep d 2.

Another aspect of the present invention relates to the use of an allergen derivative or an allergen composition according to the present invention for the preparation of an allergen specific immunotherapy medicament.

Yet another aspect of the present invention relates to the use of an allergen derivative or an allergen composition according to the present invention for the preparation of a medicament for the active immunisation.

Another aspect of the present invention relates to the use of an allergen derivative or an allergen composition according to the present invention for the preparation of a medicament for the prophylactic immunization.

Said medicament further contains preferably adjuvants, diluents, preservatives or mixtures thereof.

According to a preferred embodiment of the present invention the medicament comprises 10 ng to 1 g, preferably 100 ng to 10 mg, especially 0.5 μg to 200 μg of said recombinant allergen derivative. Preferred ways of administration include all standard administration regimes described and suggested for vaccination in general and allergy immunotherapy specifically (orally, transdermally, intraveneously, intranasally, via mucosa, etc). The present invention includes a method for treating and preventing allergy by administering an effective amount of the pharmaceutical preparations according to the present invention.

Another aspect of the present invention relates to a method for producing an allergen derivative according to the present invention, characterized by the following steps:
providing a DNA molecule encoding an allergen derivative according to the present invention,
transforming a host cell with said DNA molecule and
expressing said derivative in said host cell and isolating said derivative.

According to a preferred embodiment of the present invention said host is a eukaryotic cell, preferably a yeast or a plant cell, or a prokaryotic cell, preferably Escherichia coli.

Preferably, said host is a host with high expression capacity. As used herein, a “host with high expression capacity” is a host which expresses a protein of interest in an amount of at least 1 mg/l culture, preferably of at least 5 mg/l, more preferably of at least 10 mg/l, most preferably of at least 20 mg/l. Of course, the expression capacity depends also on the selected host and expression system (e.g. vector). Preferred hosts according to the present invention are E. coli, Pichia pastoris, Bacillus subtilis, pant cells (e.g. derived form tabacco) etc.

Of course, the allergen derivatives according to the present invention can also be produced by any other suitable method, especially chemical synthesis or semi-chemical synthesis.

The present invention is further illustrated by the following figures and examples, however, without being restricted thereto.

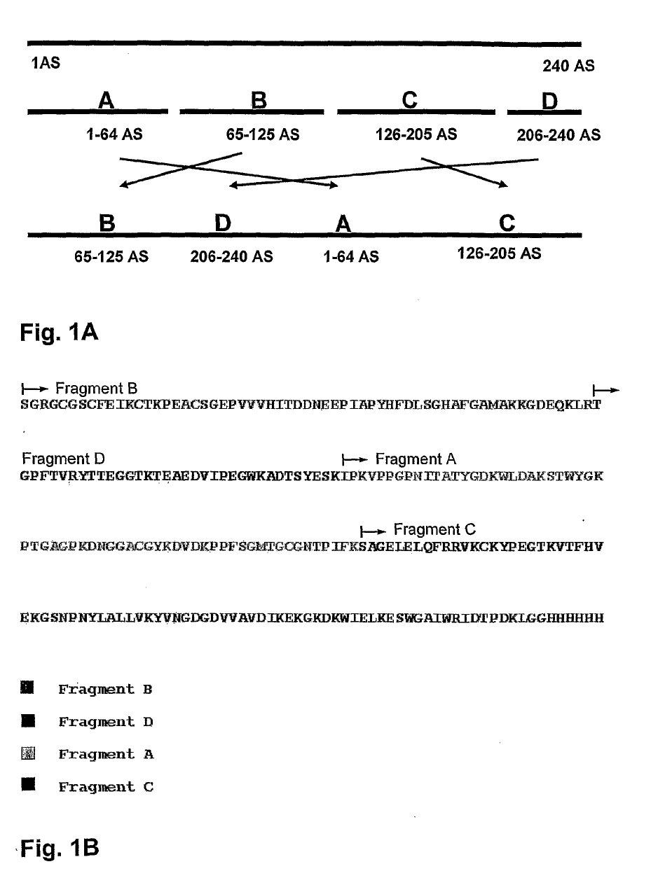

FIG. 1A shows the construction of a hypoallergenic Phl p1 derivative according to the present invention.

FIG. 1B shows the amino acid sequence of the Phl p1 mosaic protein according to the present invention (SEQ ID NO: 1).

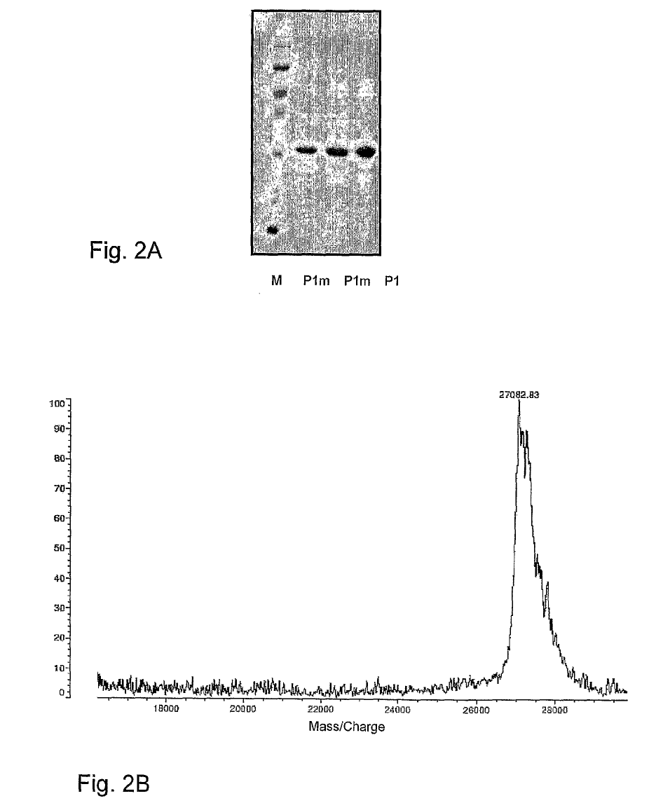

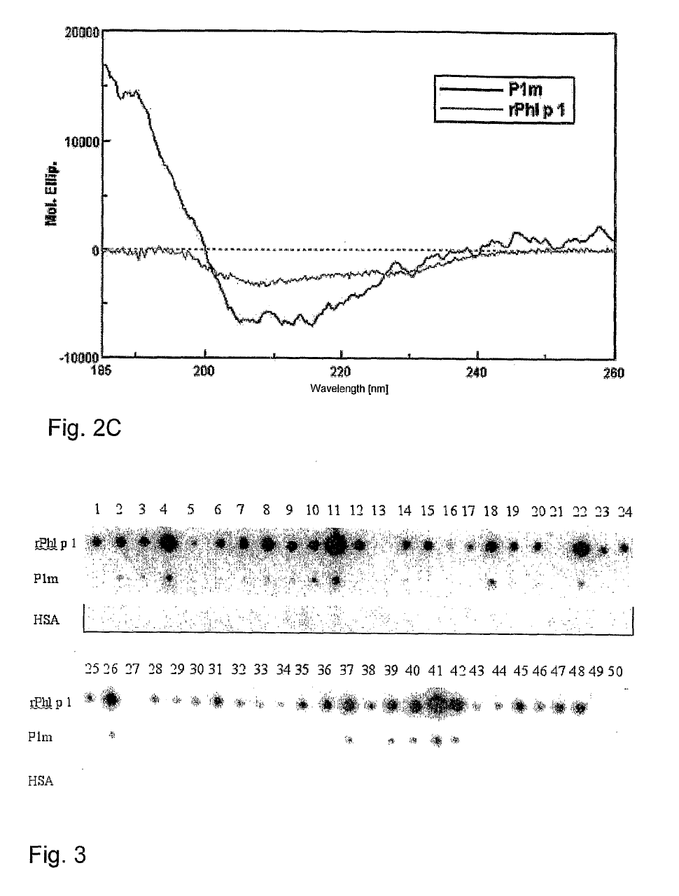

FIG. 2A shows a Coomassie-stained SDS-PAGE of P1m which has been purified to >90% purity.

FIG. 2B shows a mass spectroscopical analysis of P1m Laser desorption mass spectra were acquired in a linear mode with a TOF Compact MALDI II instrument (Kratos, UK) (piCHEM, Austria).

FIG. 2C shows a circular dichroism (CD) analysis of P1m. Far UV CD spectra were collected on a Jasco J-810 spectropolarimenter (Jasco, Easton, Md.) at room temperature, at final protein concentrations of 46 μM for P1m and 12 μM for recombinant Phl p 1 in 0.001 cm and 0.05 cm path-length quartz cuvettes, respectively. Three independent measurements were recorded and averaged for each spectral point. The final spectra were baseline corrected by substracting the corresponding buffer spectra obtained under identical conditions. Results were expressed as the mean residue ellipticity [θ] at a given wavelength.

FIG. 3 shows IgE-binding to membrane-bound recombinant allergens rPhl p 1, P1m, and HSA. Sera from 49 grass pollen allergic patients and one serum from a non-atopic donor (n=50) were incubated with membrane-bound recombinant allergens rPhl p 1, P1m, and HSA. Bound IgE was detected with ²⁵I-labelled anti-human IgE antibodies.

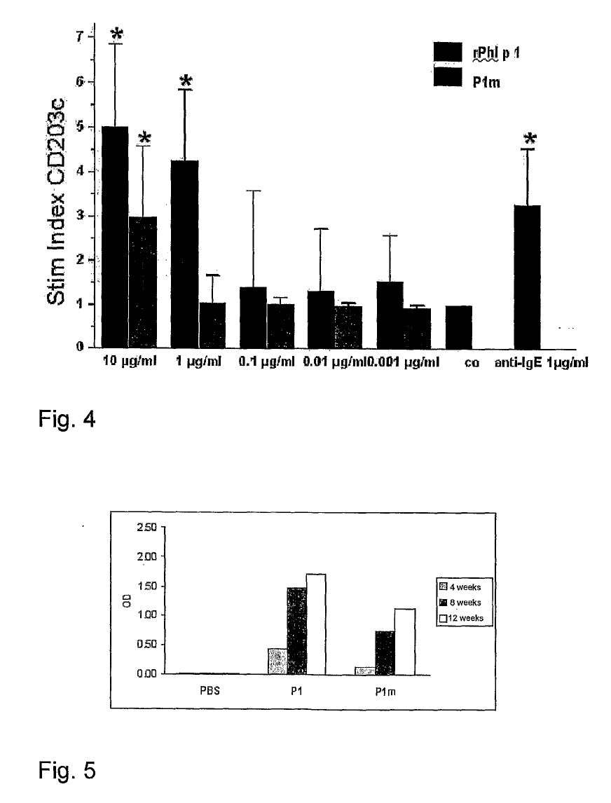

FIG. 4 shows a comparison of CD203c expression when exposing a sample of five Phl p1 allergic individuals to rPhl p1 and P1m.

FIG. 5 shows the induction of IgG1 response in mouse exposed to rPhl p1 and P1m.

### EXAMPLES

### Characterization of a Hypoallergenic Phl p 1 Mosaic (P1m) Protein

### Example 1

### Construction of a Hypoallergenic Phl p 1 Mosaic (P1m) Protein

For the construction of a recombinant hypoallergenic Phl p 1 mosaic, cDNAs coding for four Phl p 1 fragments have been amplified. Fragments A (aa 1-64), B (aa 65-125), C (aa 126-205) and D (aa 206-240) are shown in FIG. 1A. The described cDNA fragments have been assembled in the order B-D-A-C by “gene-soeing” (Horton et al., 1999). In the first PCR reactions cDNAs for fragments A (primers AF: 5′ATC CCC AAG GTT CCC 3′ and AR: 5′CAG CTC GCC GGC GCT CTT GAA GAT GGG 3′), B (primers BF: 5′C TCC TCC CAT ATG TCC GGA CGC GGC 3′ and BR: 5′GGT GAA GGG GCC CGT GCG CAG CTT CTG 3′), C (primers CF: 5′AGC GCC GGC GAG CTG 3′ and CR: 5′C GGG ATC CTA ATG ATG ATG ATG ATG ATG GGC GGC GAG CTT GTC GGG AGT GTC 3′), and D (primers DF: 5′ACG GGC CCC TTC ACC 3′ and DR: 5′GGG AAC CTT GGG GAT CTT GGA CTC GTA 3′) were obtained. In the following first SOEing-reaction the gel-purified PCR products were used as templates to obtain cDNAs coding for fragments BD (using primers BF and DR) and AC (using primers AF and CR). In the subsequent second SOEing-reaction the gel-purified fragments BD and AC were used as templates to obtain the PCR product coding for BDAC by using primers BF and CR (schematically represented in FIG. 1A).

The resulting cDNA construct coding for BDAC was inserted into the NdeI/BamHI restriction site of plasmid pET17b (Novagen, Madison, Wis.). At the C-terminus of the BDAC construct two glycines are followed by a hexahistidine-tag which allows the purification of the mosaic protein by Ni²⁺ affinity chromatography (FIG. 1B). The correct sequence of the cDNA coding for the Phl p 1 mosaic (P1m) was confirmed by sequencing.

The resulting molecule retains the entire primary sequence (Laffer et. al., 1994) and T cell epitopes (Schenk et al., 1995).

### Example 2

### Biochemical Characterization of P1m

Expression and Purification of P1m

The P1m construct was transformed into E. coli BL21 (DE3) (Stratagene, Australia) and expressed in LB-medium supplemented with 100 mg/l ampicillin. Transformed cells were grown at 37° C. to an OD600=0.9 and expression of the recombinant P1m was induced by addition of 1 mM isopropyl-β-thiogalactopyranoside (IPTG). Incubation was continued for 4 h under the same conditions and thereafter the cells were harvested by centrifugation. Recombinant P1m was purified from the insoluble pellet fraction using denaturing conditions. Cells were lysed by stirring for 60 min. in 8 M urea, 100 mM NaH₂PO₄, 10 mM Tris, pH 8.0. After centrifugation at 14,000×g for 30 min, the supernatant was loaded onto a Ni-NTA column. Recombinant P1m eluted in 8 M urea, 100 mM NaH₂PO₄, 10 mM Tris, pH 4.5 and was renatured by stepwise dialysis for at least 4 h each step against 100 mM NaH₂PO₄, 10 mM Tris, pH 8.0, containing 6, 4, 3, 2, 1, and 0.5 M urea. The protein was finally dialyzed against 10 mM Tris, 100 mM NaCl, pH 8.0 and concentrated using an Amicon centricon YM.-3 concentrator.

Protein purity was confirmed by SDS-PAGE. The protein concentration of the purified sample was estimated by UV absorption at 280 nm. The molar extinction coefficient of the protein was calculated from the tyrosine and tryptophan content (Gill et al., 1989).

P1m shows a migration pattern comparable to recombinant Phl p 1 (see FIG. 2A), which is in agreement with the molecular weight calculated according to the deduced amino acid sequence of the protein.

The molecular mass of P1m was determined by mass spectrometry to be 27 082 Dalton which is in agreement with the predicted molecular weight of the protein (see FIG. 2B).

Purified P1m was analyzed by circular dichroism analysis and compared to recombinant Phl p 1 to determine their secondary structural content (see FIG. 2C). Unexpectedly, P1m is a folded molecule with a minimum at 207 and 215 nm and a maximum at 195 nm suggesting a substantial amount of P-secondary structure. P1m, which represents an artificial allergen protein, exhibits a CD spectrum that differs to the folded version of recombinant Phl p 1, expressed in baculovirus-infected insect cells (Ball et al.). In comparison, E. coli-expressed recombinant Phl p 1 exhibited the spectrum of an unfolded protein, a fact that has been described recently (Ball et al., 2005).

### Example 3

### Reduction of IgE-Binding Capacity of P1m

The IgE reactivity of P1m was determined and compared to rPhl p 1 by dot-blot experiments under conditions of antigen excess (Niederberger et al., 1998). Three μg of the purified recombinant proteins were dotted onto nitrocellulose strips and incubated with sera from 49 Phl p 1 allergic patients. Bound IgE antibodies were detected with ¹²⁵I-labelled anti-human IgE antibodies and quantified by γ-counting (Wallac, Finland) (Ball et al., 1999).

The IgE reactivity of P1m determined under nondenaturing conditions was strongly reduced compared to the IgE binding capacity of rPhlp1 (FIG. 3). The quantification of P1m-bound IgE antibodies showed a mean reduction of the IgE-binding capacity of 86.5% of P1m compared to rPhl p 1 (Table 1).

FIG. 3 shows IgE-binding to membrane-bound recombinant allergens rPhl p 1, P1m, and HSA. Sera from 49 grass pollen allergic patients and one serum from a non-atopic donor (n=50) were incubated with membrane-bound recombinant allergens rPhl p 1, P1m, and HSA. Bound IgE was detected with ¹²⁵I-labelled anti-human IgE antibodies.

TABLE 1
Serum IgE reactivity of rPhl p 1 and P1m. Dotted
proteins were exposed to sera from 29 grass pollen-allergic patients.
Bound IgE antibodies were detected with ¹²⁵I-labelled
anti-human IgE antibodies and quantified by γ-counting.

| | |
|---|---|
| IgE Binding (cpm) | % Reduction of |

| Patient | rP1 | P1m | IgE-binding (cpm) |
|---|---|---|---|
| 1 | 855 | 31 | 96.4 |
| 2 | 604 | 209 | 65.4 |
| 4 | 252 | 45 | 82.1 |
| 5 | 724 | 47 | 93.5 |
| 6 | 1508 | 171 | 88.7 |
| 7 | 1133 | 303 | 73.3 |
| 8 | 247 | 37 | 85 |
| 9 | 1448 | 356 | 75.4 |
| 10 | 1676 | 213 | 87.3 |
| 11 | 17971 | 847 | 95.3 |
| 12 | 342 | 90 | 73.7 |
| 13 | 1539 | 77 | 95 |
| 14 | 413 | 58 | 86 |
| 15 | 105 | 28 | 73.3 |
| 16 | 252 | 19 | 92.5 |
| 17 | 300 | 27 | 91 |
| 18 | 987 | 70 | 92.9 |
| 19 | 623 | 34 | 94.5 |
| 20 | 687 | 47 | 93.2 |
| 21 | 50 | 8 | 84 |
| 22 | 694 | 42 | 93.9 |
| 23 | 1825 | 173 | 90.5 |
| 24 | 9681 | 386 | 96 |
| 25 | 1696 | 299 | 82.4 |
| 26 | 3230 | 295 | 90.9 |
| 27 | 183 | 25 | 86.3 |
| 28 | 763 | 89 | 88.3 |
| 29 | 179 | 40 | 77.7 |
| 30 | 0 | 0 | — |
| Mean | | | 86.5% |

### Example 4

### P1m Exhibits Reduced Allergenic Activity

Basophil Activation Measured by CD203c Expression

Peripheral blood was obtained from 5 allergic donors after informed consent was given. Blood was collected in heparinized tubes. Blood aliquots (100 μl) were incubated with serial dilutions of P1m and rPhl p 1 (10⁻³ to 10 μg/ml), anti-IgE antibody (1 μg/ml) (Immunotech, Marseille, France) or buffer (phosphate-buffered saline=PBS) for 15 minutes at 37° C. After incubation, cells were washed in PBS containing 20 mM EDTA. Cells were then incubated with 10 μl of PE-conjugated CD203c mAb 97A6 (Immunotech, Marseille, France) for 15 minutes at room temperature (RT). Thereafter, samples were subjected to erythrocyte lysis with 2 ml FACS™ Lysing Solution (Becton Dickinson, San Jose, Calif.). Cells were washed, resuspended in PBS, and analyzed by two-color flow cytometry on a FACScan (Becton Dickinson), using Paint-a-Gate Software. Allergen-induced upregulation of CD203c was calculated from mean fluorescence intensities (MFIs) obtained with stimulated (MFIₛₜᵢₘ) and unstimulated (MFI_{control}) cells, and expressed as stimulation index (MFIₛₜᵢₘ: MFI_{control}).

The allergenic activity of P1m was analyzed by measuring CD203c expression on blood basophils of five Phl p 1 allergic patients. As is depicted in FIG. 4, CD203c expression is significantly (p<0.05) upregulated upon incubation with rPhl p 1 at a protein concentration of 1 μg/ml, while no expression of CD203c is induced with P1m. Only when the concentration of P1m is increased to 10 μg/ml, expression of CD203c is upregulated. Thus, Phi p 1 exhibits a tenfold allergenic activity when compared to P1m.

### Example 5

### IgG Antibodies Induced by Immunization with P1m Inhibit Patients' IgE Binding to rPhl p 1

Immunization of Rabbits

Rabbits were first immunized with 200 μg P1m and rPhl p 1 using CFA, and 100 μg of the immunogens for the following booster injections using IFA (first booster injection after 4 wk; a second booster injection with incomplete adjuvant was given after 7 wk) (Charles River Breeding Laboratories, Kisslegg, Germany). Rabbits were bled 8 wk after the first immunization.

Inhibition of Allergic Patients' IgE Binding to rPhl p 1 by P1m-Induced IgG

The ability of P1m- and rPhl p 1-induced rabbit IgG to inhibit the binding of allergic patients' IgE to rPhl p 1 was tested by ELISA competition assay (Focke et al., 2001). ELISA plates (Nunc Maxisorp, Denmark) were coated with 1 μg/ml of rPhl p 1 and preincubated with 1/100 dilutions of the rPhl p 1 and P1m antisera, and, for control purposes, the corresponding preimmune sera. After washing, plates were incubated with 1/10 diluted sera from forty three Phl p 1-sensitized grass pollen allergic patients. Bound IgE Abs were detected with HRP-coupled goat anti-human IgE Abs (KPL, Gaithersburg, Md.), diluted 1/2500. The percentage of inhibition of IgE binding achieved by preincubation with the anti-Phl p 1 and anti-P1m antisera was calculated as follows: percentage of inhibition of IgE binding=100−OD_{I}/OD_{P}×100. OD_{I} and OD_{P} represent the extinctions after pre-incubation with the rabbits' immune sera and the corresponding preimmune sera, respectively.

The ability of P1m to inhibit patients' IgE binding to rPhl p 1 is shown as percentage reduction in Table 2. The strongest inhibition of patients' IgE binding to rPhl p 1 ranging from 0 to 89% (52% mean reduction) was achieved with anti-P1m Abs whereas inhibition with anti-Phl p 1 Abs was in the range of 4 to 75% (43% mean).

TABLE 2
Rabbit antisera raised against rPhl p 1 and P1m inhibit
binding of grass pollen-allergic patients IgE to rPhl p 1.
ELISA plate-bound rPhl p 1 and P1m was preincubated with rabbit
anti-rPhl p1 and anti-P1m antisera. The percentages reduction of
IgE-binding obtained for fourty three grass pollen allergic patients
are displayed.
% Reduction of IgE-binding to rPhl p1 by
P1m induced rabbit IgG

OD values:

| Patient | rαP1 | rαP1m |
|---|---|---|
| 1 | 37 | 50 |
| 2 | 44 | 61 |
| 3 | 32 | 49 |
| 4 | 50 | 70 |
| 5 | 4 | 34 |
| 6 | 44 | 52 |
| 7 | 55 | 75 |
| 8 | 27 | 38 |
| 9 | 51 | 55 |
| 10 | 28 | 0 |
| 11 | 32 | 13 |
| 12 | 36 | 24 |
| 13 | 40 | 36 |
| 14 | 20 | 14 |
| 15 | 23 | 51 |
| 16 | 39 | 60 |
| 17 | 41 | 56 |
| 18 | 25 | 41 |
| 19 | 42 | 55 |
| 20 | 43 | 58 |
| 22 | 62 | 56 |
| 23 | 55 | 68 |
| 24 | 46 | 50 |
| 26 | 35 | 32 |
| 27 | 31 | 24 |
| 28 | 70 | 77 |
| 29 | 28 | 38 |
| 30 | 58 | 65 |
| 31 | 43 | 57 |
| 32 | 52 | 56 |
| 33 | 56 | 66 |
| 34 | 59 | 66 |
| 36 | 69 | 80 |
| 37 | 49 | 70 |
| 38 | 72 | 81 |
| 39 | 37 | 61 |
| 40 | 68 | 78 |
| 41 | 23 | 37 |
| 42 | 75 | 89 |
| 43 | 49 | 68 |
| 44 | 72 | 81 |
| 45 | 72 | 85 |
| 47 | 9 | 23 |
| mean | 43 | 52 |

### Example 6

### Immunogenicity of P1m

To investigate whether the Phl p 1 mosaic protein induces an allergen-specific IgG response in vivo, 6 weeks old female BALB/c mice (Charles River Breeding Laboratories) were immunized subcutaneously. Groups of 5 mice were immunized with 5 μg P1m, rPhl p 1 and, for control purposes, with PBS only adsorbed to Al(OH)₃ (Alu-Gel-S, Serva, Germany) (Vrtala et al., 1998). Mice were immunized three times (day 1, 28 and 56) and bled from the tail veins in 4-week intervals, and sera stored at −20° C. until analysis.

IgG1 responses to rPhl p 1 were measured by ELISA (Vrtala et al., 1998). ELISA plates (Nunc Maxisorp, Denmark) were coated with 5 μg of P1m and incubated with 1:1000 diluted mouse sera. Bound IgG1 antibodies were detected with a 1:1000 diluted monoclonal rat anti-mouse IgG1 (Pharmingen, Calif.) and a 1:200 diluted HRP-labeled sheep anti-rat antiserum (Amersham, UK).

As is demonstrated in FIG. 5, the P1m induced IgG1 response to rPhl p 1 is of almost the magnitude as that induced by immunization with rPhl p 1.

FIG. 5. Immunogenicity of P1m. Mean IgG₁ response of 5 mice immunized with PBS, rPhl p1 or P1m or (x-axis) displayed as OD values (y-axis) 4, 8 weeks or 12 weeks after immunization.

### REFERENCES

Ball, T., et al. (2005) FEBS J. 272:217-227.
Ball, T., et al. (1999) FASEB J. 13:1277.
Focke, M., et al. (2001) FASEB J. 15:2042
Gill, S. C., and P. H. Hippel. (1989) Anal. Biochem. 182: 319.
Hauswirth, A W., et al. (2002) J Allergy Clin Immunol 110:102.
Horton, R. M. (1999) Mol. Biotechnol. 2:93.
Laffer, S., et al. (1994) J. Allergy Clin. Immunol. 94:689.
Niederberger, V., et al. (1998) J. Allergy Clin. Immunol. 101:258.
Schenk, S., et al. (1995) J. Allergy Clin. Immunol. 96:986.
Vrtala, S., et al. (1998) J. Immunol. 160: 6137.

1-31. (canceled)

32. A method for producing derivatives of wild-type protein allergen Phl p 1 with reduced allergenic activity compared to the wild-type allergen, comprising the following steps:
providing a wild-type protein allergen Phl p 1;
fragmenting said wild-type protein allergen into at least three fragments, wherein at least one fragment of said at least three fragments comprises at least one T-cell epitope and said at least three fragments have a reduced allergenic activity or lack allergenic activity; and
rejoining said at least three fragments in an order differing from the order of the fragments in the wild-type allergen.

33. The method according to claim 32, wherein the reduction in allergenic activity is measured by a reduction of IgE binding capacity of at least 10%, compared to the wild-type allergen.

34. The method according to claim 32, wherein the reduction in allergenic activity is measured by lack of binding of IgE antibodies of allergen sensitised a patient's sera to a dot blot of said derivative or by a basophil release assay.

35. The method according to claim 32, wherein the at least three fragments comprise amino acid residues 25 to 39, amino acid residues 34 to 45, amino acid residues 73 to 84, amino acid residues 91 to 102, amino acid residues 100 to 111, amino acid residues 109 to 133, amino acid residues 121 to 135, amino acid residues 127 to 138, amino acid residues 157 to 168, amino acid residues 169 to 183 or amino acid residues 226 to 240 of Phl p 1.

36. The method according to claim 32, wherein the at least three fragments are selected from the group consisting of
amino acid residues 1 to 64 (A),
amino acid residues 65 to 125 (B),
amino acid residues 126 to 205 (C) and
amino acid residues 206 to 240 (D) of Phl p 1.

37. The method according to claim 36, wherein the order of said fragments in the allergen derivative is B-D-A-C.

38. The method according to claim 32, wherein said derivatives are combined with a pharmaceutically acceptable excipient and finished to a pharmaceutical preparation.

39. The method according to claim 1, wherein said derivatives are combined with a suitable vaccine adjuvant and finished to a pharmaceutically acceptable vaccine preparation.

40. The method according to claim 39, wherein said derivatives are combined with at least one further allergen to a combination vaccine.

41. The method according to claim 40, wherein said further allergen is a wild-type allergen, a mixture of wild-type allergens, recombinant wild-type allergens, derivatives of wild-type protein allergens or mixtures thereof.

42. The method according to claim 32, wherein said preparation further contains an allergen extract.

43. The method according to claim 40, wherein said further allergen is selected from the group consisting of the major birch pollen allergens, Bet v 1, Bet v 4, the major timothy grass pollen allergens, Phl p 2, Phl p 5, Phl p 6, Phl p 7, the major house dust mite allergens, Der p 1, Der p 2, the major cat allergen Fel d 1, the major bee allergens, the major wasp allergens, profilins, Phl p 12, storage mite allergens, and Lep d 2.

44. An allergen derivative obtainable by the method according to claim 32.

45. An allergen derivative of wild-type protein allergen Phl p 1, wherein said derivative contains at least three fragments of said wild-type protein allergen fused to each other in an order differing from the order of the fragments in the wild-type allergen, wherein said at least three wild-type allergen fragments exhibit reduced allergenic activity or lack allergenic activity and wherein at least one of said at least three fragments comprises one or more T cell epitopes.

46. The allergen derivative according to claim 45, wherein said at least three allergen fragments comprise at least 6 amino acid residues.

47. The allergen derivative according to claim 45, wherein the at least three fragments comprise amino acid residues 25 to 39, amino acid residues 34 to 45, amino acid residues 1 to 64, amino acid residues 73 to 84, amino acid residues 91 to 102, amino acid residues 100 to 111, amino acid residues 109 to 133, amino acid residues 121 to 135, amino acid residues 65 to 125, amino acid residues 126 to 205, amino acid residues 127 to 138, amino acid residues 157 to 168, amino acid residues 169 to 183, amino acid residues 206 to 240 or amino acid residues 226 to 240 of Phl p 1.

48. The allergen derivative according to claim 45 wherein the at least three fragments are selected from the group consisting of amino acid residues 1 to 64 (A), amino acid residues 65 to 125 (B), amino acid residues 126 to 205 (C) and amino acid residues 206 to 240 (D) of Phl p 1.

49. The allergen derivative according to claim 48, wherein the order of said fragments in the allergen derivative is B-D-A-C.

50. The allergen derivative according to claim 45 further comprising one or more further allergens selected from—wild-type allergens, a mixture of wild-type allergens, recombinant wild-type allergens, derivatives of wild-type protein allergens or mixtures thereof.

51. The allergen derivative according to claim 50, further comprising an allergen extract.

52. The allergen derivative according to claim 50, further comprising a pharmaceutically acceptable excipient.

53. The allergen derivative according to claim 50, characterised in that said composition further comprises one or more allergens selected from the group consisting of birch pollen allergens, Bet v 1, Bet v 4, timothy grass pollen allergens, Phl p 1, Phl p 2, Phl p 5, Phl p 6, Phl p 7, dust mite allergens, Der p 1, Der p 2, cat allergen Fel d 1, bee allergens, wasp allergens, profilins, Phl p 12, storage mite allergens, and Lep d 2.

54. An immunotherapy treatment comprising the steps of:
administering an allergen composition to a subject in need of immunotherapy treatment, wherein the allergen composition comprises a wild-type protein allergen derivative made by fragmenting said wild-type protein allergen into at least three fragments, wherein at least one fragment of said at least three fragments comprises at least one T-cell epitope and said at least three fragments have a reduced allergenic activity or lack allergenic activity; and
rejoining said at least three fragments in an order differing from the order of the fragments in the wild-type allergen.

55. The method of claim 54, wherein the administering provides active immunisation.

56. The method of claim 54, wherein the administering provides prophylactic immunization.

57. The method of claim 54, wherein the allergen composition further comprises adjuvants, diluents, preservatives or mixtures thereof.

58. The method of claim 54, wherein the allergen composition comprises 10 ng to 1 g of said allergen derivative.

59. The method of claim 54, wherein the allergen composition comprises 100 ng to 10 mg of said allergen derivative.

60. The method of claim 54, wherein the allergen composition comprises 0.5 μg to 200 μg of said allergen derivative.

61. A method for producing an allergen derivative comprising the steps of:
providing a DNA molecule encoding an allergen derivative comprising at least three fragments of a wild-type protein allergen fused to each other in an order differing from the order of the fragments in the wild-type allergen, wherein said at least three wild-type allergen fragments exhibit reduced allergenic activity or lack allergenic activity and wherein at least one of said at least three fragments comprises one or more T cell epitopes;
transforming a host cell with said DNA molecule;
expressing said derivative in said host cell; and
isolating said derivative.

62. The method according to claim 61, wherein said host is a eukaryotic cell, a yeast cell, a plant cell, a prokaryotic cell, Escherichia coli cell or a combination thereof.

63. The method according to claim 61, characterized in that the allergen derivative is produced by chemical synthesis.


SEQUENCE LISTING (9 sequences)

SEQ ID NO: 1 - protein, 248 aa, Artificial
  Phl p 1 Mosaic Protein
SGRGCGSCFEIKCTKPEACSGEPVVVHITDDNEEPIAPYHFDLSGHAFGAMAKKGDEQKLRTGPFTVRYTTEGGTKTEAEDVIPEGWKADTSYESKIPKVPPGPNITATYGDKWLDAKSTWYGKPTGAGPKDNGGACGYKDVDKPPFSGMTGCGNTPIFKSAGELELQFRRVKCKYPEGTKVTFHVEKGSNPNYLALLVKYVNGDGDVVAVDIKEKGKDKWIELKESWGAIWRIDTPDKLGGHHHHHH

SEQ ID NO: 2 - DNA, 15 bp, Artificial
  Primer
atccccaaggttccc

SEQ ID NO: 3 - DNA, 27 bp, Artificial
  Primer
cagctcgccggcgctcttgaagatggg

SEQ ID NO: 4 - DNA, 25 bp, Artificial
  Primer
ctcctcccatatgtccggacgcggc

SEQ ID NO: 5 - DNA, 27 bp, Artificial
  Primer
ggtgaaggggcccgtgcgcagcttctg

SEQ ID NO: 6 - DNA, 15 bp, Artificial
  Primer
agcgccggcgagctg

SEQ ID NO: 7 - DNA, 52 bp, Artificial
  Primer
cgggatcctaatgatgatgatgatgatgggcggcgagcttgtcgggagtgtc

SEQ ID NO: 8 - DNA, 15 bp, Artificial
  Primer
acgggccccttcacc

SEQ ID NO: 9 - DNA, 27 bp, Artificial
  Primer
gggaaccttggggatcttggactcgta